SEQUENCE LISTING (25 sequences)

SEQ ID NO: 1 - protein, 2078 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 2 - protein, 525 aa, synthetic construct
MPSAFSYVRFSSGKQAKGSSEHRQRAMLGQWLEQHPSFTLSDLRFEDLGRSGFSGEHLDHGLGQLLAAIDSGAIKSGDVILVEAVDRIGRLEPLEMLPLFSRIVKAGVSVITLEDGHVYDRSSVNETSLFLLVAKIQQAHEYSNRLSRRINASYTARREKAKAGLGIKRETPVWLTTDGQLVPHVAPHIAQAFQDYADGLGERRICRKLRESGLEEFSKTNATTVRRWLKNRTAIGYWNDIPDVYPHVVDPALFYQVQQRLDAPKVDRAKPSAHYLTGLVKCAVCGRNYNYKQRKHTDPAMLCTSRARLAGEGCSNSKTYPVIVLDQVRKLTSLPFLQHAMESASSQADPSSQRLAVIDGEIGELSRKISEATKALLVLGFTPEIQESLEQLKTAREALEEERATLLLPQAEKLTTAQLEAFSNGLLDDEPMKLNHVLQTAGYSMVVHPDGSIDVDGKRFVYEGASRKEKVYKLRLIGEDKQWSLPILTPQMATYKSLFMAAVRLPGDPSEEELRRFEEAKHSER

SEQ ID NO: 3 - protein, 18 aa, synthetic construct
GGSGGSGGSGGSGGSGGS

SEQ ID NO: 4 - protein, 2679 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKVGGSGGSGGSGGSGGSGGSPKKKRKVYPYDVPDYAGSMPSAFSYVRFSSGKQAKGSSEHRQRAMLGQWLEQHPSFTLSDLRFEDLGRSGFSGEHLDHGLGQLLAAIDSGAIKSGDVILVEAVDRIGRLEPLEMLPLFSRIVKAGVSVITLEDGHVYDRSSVNETSLFLLVAKIQQAHEYSNRLSRRINASYTARREKAKAGLGIKRETPVWLTTDGQLVPHVAPHIAQAFQDYADGLGERRICRKLRESGLEEFSKTNATTVRRWLKNRTAIGYWNDIPDVYPHVVDPALFYQVQQRLDAPKVDRAKPSAHYLTGLVKCAVCGRNYNYKQRKHTDPAMLCTSRARLAGEGCSNSKTYPVIVLDQVRKLTSLPFLQHAMESASSQADPSSQRLAVIDGEIGELSRKISEATKALLVLGFTPEIQESLEQLKTAREALEEERATLLLPQAEKLTTAQLEAFSNGLLDDEPMKLNHVLQTAGYSMVVHPDGSIDVDGKRFVYEGASRKEKVYKLRLIGEDKQWSLPILTPQMATYKSLFMAAVRLPGDPSEEELRRFEEAKHSER

SEQ ID NO: 5 - DNA, 6234 bp, synthetic construct
  source 1..6234: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagaaagctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaagagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcaccctgaacattgaagacgagtatagactgcatgaaacaagcaaggaacccgacgtgtccctgggctccacctggctgtccgactttccccaggcctgggccgagacaggaggaatgggcctggccgtgcggcaggcacccctgatcatccctctgaaggccacctctacacccgtgagcatcaagcagtaccctatgtctcaggaggccagactgggcatcaagcctcacatccagaggctgctggaccagggcatcctggtgccatgccagagcccctggaacacaccactgctgcccgtgaagaagccaggcaccaatgactatagacccgtgcaggatctgagagaggtgaacaagagggtggaggatatccaccccaccgtgcccaacccttacaatctgctgtccggcccccccccttctcaccagtggtatacagtgctggacctgaaggatgccttcttttgtctgagactgcaccctaccagccagccactgttcgcctttgagtggagggaccctgagatgggcatctctggccagctgacctggacacgcctgcctcagggcttcaagaatagcccaacactgtttaacgaggccctgcaccgcgacctggcagatttccggatccagcacccagatctgatcctgctgcagtacgtggacgatctgctgctggccgccaccagcgagctggattgccagcagggaacacgcgccctgctgcagaccctgggaaacctgggatatagggcatccgccaagaaggcccagatctgtcagaagcaggtgaagtacctgggctatctgctgaaggagggccagagatggctgacagaggccaggaaggagacagtgatgggccagccaacacccaagaccccaagacagctgagggagttcctgggcaaagcaggattttgcaggctgttcatcccaggattcgcagagatggcagcacctctgtacccactgaccaagccgggcaccctgtttaattggggccctgaccagcagaaggcctatcaggagatcaagcaggccctgctgacagcaccagccctgggcctgccagacctgaccaagcctttcgagctgtttgtggatgagaagcagggctacgccaagggcgtgctgacccagaagctgggaccatggagacggcccgtggcctatctgtccaagaagctggacccagtggcagcaggatggccaccatgcctgaggatggtggcagcaatcgccgtgctgacaaaggatgccggcaagctgaccatgggacagccactggtcatcctggcaccacacgcagtggaggccctggtgaagcagcctccagatcgctggctgtctaacgcccggatgacacactaccaggccctgctgctggacaccgatcgcgtgcagtttggccctgtggtggccctgaatccagccaccctgctgcctctgccagaggagggcctgcagcacaactgtctggacatcctggcagaggcacacggaacaaggccagacctgaccgatcagcccctgcctgacgccgatcacacatggtataccgatggaagctccctgctgcaggagggccagaggaaggcaggagcagcagtgaccacagagacagaagtgatctgggccaaggccctgccagcaggcacatccgcccagcgggccgagctgatcgccctgacccaggccctgaagatggccgagggcaagaagctgaacgtgtacacagactccagatatgccttcgccaccgcacacatccacggagagatctacaggcgccggggctggctgacctctgagggcaaggagatcaagaacaaggatgagatcctggccctgctgaaggccctgtttctgcccaagcggctgagcatcatccactgtcctggacaccagaagggacactccgccgaggcaaggggcaatcggatggccgaccaggccgccagaaaggctgctattactgaaactcccgacacttccactctgctgattgaaaactcctcccct

SEQ ID NO: 6 - DNA, 54 bp, synthetic construct
  source 1..54: mol_type = other DNA
ggggggtcaggtggatccggcggaagtggcggatccggtggatctggcggcagt

SEQ ID NO: 7 - DNA, 1575 bp, synthetic construct
  source 1..1575: mol_type = other DNA
atgccttctgccttctcctatgtgcgcttcagtagcggaaagcaagccaaaggcagctctgagcacaggcagagagcgatgctaggccagtggctagagcagcacccttctttcaccctgtctgacctgagatttgaagacttgggcaggtccggcttcagcggcgaacacctggaccatggcctgggacagctcctggcagcaatagacagtggtgctattaagagcggtgacgtgatcctggtggaggctgtagaccggatcggccgtctggaacccctggagatgctgccactgtttagcaggatcgtcaaggcaggagtttctgttatcaccctggaggatggacacgtgtatgacagaagcagtgtgaatgaaacctccctcttcctgctcgtggcaaagatccagcaggcccatgagtactctaacagactcagcaggagaattaatgcctcctacactgctcggcgagagaaagccaaagcgggcctcggcatcaagcgggaaacccctgtgtggcttaccacagatggccaactcgtgcctcacgtggccccccacatcgctcaggctttccaggattacgctgatgggctgggagaaagaagaatctgcaggaagttgagagaatcaggcttagaggagtttagtaagacgaacgccaccacagtgcggcgctggctgaaaaaccgaactgccatcggttactggaatgacatccctgatgtctaccctcacgtcgtggacccagctctgttctaccaggtgcaacaaagactggatgcgccgaaagtggacagggccaagccttctgcccactacctgacgggtctggtgaagtgtgctgtctgtggccgcaactacaactataagcagagaaaacacacagacccagccatgctctgcacctcccgtgcgaggctggccggagaaggctgctccaactccaaaacatacccagtgatcgtgctcgaccaggtgcggaagctgaccagcctgcccttcttgcaacacgcaatggagagcgcctcgagccaggctgaccccagctcccagcgactggccgtgatcgacggcgagattggcgagctgagtcggaagatctccgaggcaaccaaggccctgctggtcctaggcttcacccctgagatacaagagtcgctggaacagctgaagacagcaagagaagctctagaggaggagcgggccactctgctcctcccccaggcagaaaagcttaccacagcccagttggaggctttctcaaacgggctgctggatgatgagcccatgaagctcaaccatgtgttgcagacagcgggatactccatggtggttcaccctgacggcagcatcgacgtggatggtaagagatttgtgtatgaaggggccagcagaaaggaaaaggtgtacaaactgcggttgattggagaggataaacagtggagcctgcccatcttaaccccccagatggccacctataagagccttttcatggcagctgtcagactgcctggagatccttctgaggaagagttacggagatttgaggaggccaagcatagtgaacgg

SEQ ID NO: 8 - DNA, 8040 bp, synthetic construct
  source 1..8040: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagaaagctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaagagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcaccctgaacattgaagacgagtatagactgcatgaaacaagcaaggaacccgacgtgtccctgggctccacctggctgtccgactttccccaggcctgggccgagacaggaggaatgggcctggccgtgcggcaggcacccctgatcatccctctgaaggccacctctacacccgtgagcatcaagcagtaccctatgtctcaggaggccagactgggcatcaagcctcacatccagaggctgctggaccagggcatcctggtgccatgccagagcccctggaacacaccactgctgcccgtgaagaagccaggcaccaatgactatagacccgtgcaggatctgagagaggtgaacaagagggtggaggatatccaccccaccgtgcccaacccttacaatctgctgtccggcccccccccttctcaccagtggtatacagtgctggacctgaaggatgccttcttttgtctgagactgcaccctaccagccagccactgttcgcctttgagtggagggaccctgagatgggcatctctggccagctgacctggacacgcctgcctcagggcttcaagaatagcccaacactgtttaacgaggccctgcaccgcgacctggcagatttccggatccagcacccagatctgatcctgctgcagtacgtggacgatctgctgctggccgccaccagcgagctggattgccagcagggaacacgcgccctgctgcagaccctgggaaacctgggatatagggcatccgccaagaaggcccagatctgtcagaagcaggtgaagtacctgggctatctgctgaaggagggccagagatggctgacagaggccaggaaggagacagtgatgggccagccaacacccaagaccccaagacagctgagggagttcctgggcaaagcaggattttgcaggctgttcatcccaggattcgcagagatggcagcacctctgtacccactgaccaagccgggcaccctgtttaattggggccctgaccagcagaaggcctatcaggagatcaagcaggccctgctgacagcaccagccctgggcctgccagacctgaccaagcctttcgagctgtttgtggatgagaagcagggctacgccaagggcgtgctgacccagaagctgggaccatggagacggcccgtggcctatctgtccaagaagctggacccagtggcagcaggatggccaccatgcctgaggatggtggcagcaatcgccgtgctgacaaaggatgccggcaagctgaccatgggacagccactggtcatcctggcaccacacgcagtggaggccctggtgaagcagcctccagatcgctggctgtctaacgcccggatgacacactaccaggccctgctgctggacaccgatcgcgtgcagtttggccctgtggtggccctgaatccagccaccctgctgcctctgccagaggagggcctgcagcacaactgtctggacatcctggcagaggcacacggaacaaggccagacctgaccgatcagcccctgcctgacgccgatcacacatggtataccgatggaagctccctgctgcaggagggccagaggaaggcaggagcagcagtgaccacagagacagaagtgatctgggccaaggccctgccagcaggcacatccgcccagcgggccgagctgatcgccctgacccaggccctgaagatggccgagggcaagaagctgaacgtgtacacagactccagatatgccttcgccaccgcacacatccacggagagatctacaggcgccggggctggctgacctctgagggcaaggagatcaagaacaaggatgagatcctggccctgctgaaggccctgtttctgcccaagcggctgagcatcatccactgtcctggacaccagaagggacactccgccgaggcaaggggcaatcggatggccgaccaggccgccagaaaggctgctattactgaaactcccgacacttccactctgctgattgaaaactcctccccttctggcggctcaaaaagaaccgccgacggcagcgaattcgagcccaagaagaagaggaaagtcggggggtcaggtggatccggcggaagtggcggatccggtggatctggcggcagtccaaaaaagaaaagaaaagtgtatccctatgatgtccccgattatgccggttcaatgccttctgccttctcctatgtgcgcttcagtagcggaaagcaagccaaaggcagctctgagcacaggcagagagcgatgctaggccagtggctagagcagcacccttctttcaccctgtctgacctgagatttgaagacttgggcaggtccggcttcagcggcgaacacctggaccatggcctgggacagctcctggcagcaatagacagtggtgctattaagagcggtgacgtgatcctggtggaggctgtagaccggatcggccgtctggaacccctggagatgctgccactgtttagcaggatcgtcaaggcaggagtttctgttatcaccctggaggatggacacgtgtatgacagaagcagtgtgaatgaaacctccctcttcctgctcgtggcaaagatccagcaggcccatgagtactctaacagactcagcaggagaattaatgcctcctacactgctcggcgagagaaagccaaagcgggcctcggcatcaagcgggaaacccctgtgtggcttaccacagatggccaactcgtgcctcacgtggccccccacatcgctcaggctttccaggattacgctgatgggctgggagaaagaagaatctgcaggaagttgagagaatcaggcttagaggagtttagtaagacgaacgccaccacagtgcggcgctggctgaaaaaccgaactgccatcggttactggaatgacatccctgatgtctaccctcacgtcgtggacccagctctgttctaccaggtgcaacaaagactggatgcgccgaaagtggacagggccaagccttctgcccactacctgacgggtctggtgaagtgtgctgtctgtggccgcaactacaactataagcagagaaaacacacagacccagccatgctctgcacctcccgtgcgaggctggccggagaaggctgctccaactccaaaacatacccagtgatcgtgctcgaccaggtgcggaagctgaccagcctgcccttcttgcaacacgcaatggagagcgcctcgagccaggctgaccccagctcccagcgactggccgtgatcgacggcgagattggcgagctgagtcggaagatctccgaggcaaccaaggccctgctggtcctaggcttcacccctgagatacaagagtcgctggaacagctgaagacagcaagagaagctctagaggaggagcgggccactctgctcctcccccaggcagaaaagcttaccacagcccagttggaggctttctcaaacgggctgctggatgatgagcccatgaagctcaaccatgtgttgcagacagcgggatactccatggtggttcaccctgacggcagcatcgacgtggatggtaagagatttgtgtatgaaggggccagcagaaaggaaaaggtgtacaaactgcggttgattggagaggataaacagtggagcctgcccatcttaaccccccagatggccacctataagagccttttcatggcagctgtcagactgcctggagatccttctgaggaagagttacggagatttgaggaggccaagcatagtgaacggtag

SEQ ID NO: 9 - RNA, 185 nt, synthetic construct
  source 1..185: mol_type = other RNA
ggcatctgagaacccttagggttttagagctagaaatagcaagttaaaataaggctagtccgttatcaacttgaaaaagtggcaccgagtcggtgccttcgtggcgcatacgcccttcgagcatgtaggtcacggaagggttctcagacgcggttctatctagttacgcgttaaaccaactagaa

SEQ ID NO: 10 - RNA, 185 nt, synthetic construct
  source 1..185: mol_type = other RNA
gtagtatagcatggtcgagcgttttagagctagaaatagcaagttaaaataaggctagtccgttatcaacttgaaaaagtggcaccgagtcggtgcccgtgacctacatgctcgaagggcgtatgcgccacgaagcgaccatgctatacgcggttctatctagttacgcgttaaaccaactagaa

SEQ ID NO: 11 - DNA, 185 bp, synthetic construct
  source 1..185: mol_type = other DNA
ggcatctgagaacccttagggttttagagctagaaatagcaagttaaaataaggctagtccgttatcaacttgaaaaagtggcaccgagtcggtgccttcgtggcgcatacgcccttcgagcatgtaggtcacggaagggttctcagacgcggttctatctagttacgcgttaaaccaactagaa

SEQ ID NO: 12 - DNA, 185 bp, synthetic construct
  source 1..185: mol_type = other DNA
gtagtatagcatggtcgagcgttttagagctagaaatagcaagttaaaataaggctagtccgttatcaacttgaaaaagtggcaccgagtcggtgcccgtgacctacatgctcgaagggcgtatgcgccacgaagcgaccatgctatacgcggttctatctagttacgcgttaaaccaactagaa

SEQ ID NO: 13 - DNA, 104 bp, synthetic construct
  source 1..104: mol_type = other DNA
gtaacgctcttcgagaaagcagattctcatatccatcttgagtcttctttctcgcaagacaacacgaaatagacacagtctcttccctagctgtacactgagcc

SEQ ID NO: 14 - DNA, 33 bp, synthetic construct
  source 1..33: mol_type = other DNA
ccgtgacctacatgctcgaagggcgtatgcgcc

SEQ ID NO: 15 - DNA, 717 bp, synthetic construct
  source 1..717: mol_type = other DNA
gccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 16 - RNA, 39 nt, synthetic construct
  source 1..39: mol_type = other RNA
cttcgtggcgcatacgcccttcgagcatgtaggtcacgg

SEQ ID NO: 17 - RNA, 39 nt, synthetic construct
  source 1..39: mol_type = other RNA
ccgtgacctacatgctcgaagggcgtatgcgccacgaag

SEQ ID NO: 18 - DNA, 39 bp, synthetic construct
  source 1..39: mol_type = other DNA
cttcgtggcgcatacgcccttcgagcatgtaggtcacgg

SEQ ID NO: 19 - DNA, 39 bp, synthetic construct
  source 1..39: mol_type = other DNA
ccgtgacctacatgctcgaagggcgtatgcgccacgaag

SEQ ID NO: 20 - DNA, 75 bp, synthetic construct
  source 1..75: mol_type = other DNA
tttcttaagagatgtaaaattttcatgatgttttcttttttgctaaaactaaagaattattcttttacatttcag

SEQ ID NO: 21 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 22 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 23 - protein, 8 aa, synthetic construct
SGGSSGGS

SEQ ID NO: 24 - protein, 7 aa, synthetic construct
PKKKRKV

SEQ ID NO: 25 - protein, 17 aa, synthetic construct
KRTADGSEFEPKKKRKV

(19) United States
(12) Patent Application Publication
(10) Pub. No.: US 2024/0401013 A1
(43) Pub. Date: Dec. 5, 2024

(54) ENHANCED PROGRAMMABLE ADDITION VIA SITE-SPECIFIC TARGETING ELEMENTS SYSTEM USING A FUSION PROTEIN OF PRIME EDITOR PROTEIN AND PA01 INTEGRASE

(71) Applicant: UIF (UNIVERSITY INDUSTRY FOUNDATION), YONSEI UNIVERSITY, Seoul (KR)

(72) Inventors: Hyongbum Henry KIM, Seoul (KR); Hyewon JANG, Seoul (KR); Sung-Ik CHO, Seoul (KR)

(73) Assignee: UIF (UNIVERSITY INDUSTRY FOUNDATION), YONSEI UNIVERSITY, Seoul (KR)

(21) Appl. No.: 18/678,602

(22) Filed: May 30, 2024

(30) Foreign Application Priority Data
May 30, 2023 (KR) 10-2023-0069455
Apr. 30, 2024 (KR) 10-2024-0058109

(51) Int. Cl. C12N 9/22; C12N 9/12; C12N 15/11

(52) U.S. Cl. CPC C12N 9/22; C12N 9/1276; C12N 15/111; C07K 2319/09; C12N 2310/20; C12Y 207/07049

(57) ABSTRACT

An Enhanced Programmable Addition via Site-specific Targeting Elements (EPASTE) system utilizing a fusion protein of Prime Editor (PE) protein and Pa01 Integrase, and the EPASTE system enables the integration of large donor DNA into a target site of an intracellular genome with significantly superior efficiency than existing technologies, and without the need for a DNA repair process for DNA double-strand breakage.

### CROSS-REFERENCE TO RELATED APPLICATION

This application is based on and claims priority under 35 USC § 119 to Korean Patent Application Nos. 10-2023-0069455, filed on May 30, 2023, and 10-2024-0058109, filed on Apr. 30, 2024 in the Korean Intellectual Property Office, the disclosure of which is incorporated by reference herein in its entirety.

### BACKGROUND

### 1. Field

The present disclosure relates to an Enhanced Programmable Addition via Site-specific Targeting Elements (EPASTE) system using a fusion protein of Prime Editor (PE) protein and Pa01 Integrase.

### 2. Description of the Related Art

Programmable site-specific integration of large donor DNA without DNA double-strand break repair processes remains an unsolved issue, and research is ongoing to address this using Prime Editor (PE) proteins. Currently known technologies capable of programmable site-specific integration include PASTE v3, PASTE v4, and Twin-PE-mediated KI. The PASTE v3 and Twin-PE-mediated KI systems use a protein that is a fusion of BxbI integrase with Prime Editor protein, while the PASTE v4 system uses a protein that is a fusion of BceI integrase with Prime Editor protein. However, even with these existing technologies, the efficiency of integrating foreign genes, i.e., donor DNA, into target sites of the intracellular genome remains very low, and there is a need for the development of technologies to increase the efficiency of site-specific foreign gene integration.

Accordingly, the inventors of the present disclosure have developed the Enhanced Programmable Addition via Site-specific Targeting Elements (EPASTE) system, utilizing a fusion protein of Prime Editor protein and Pa01 Integrase, and have confirmed that the EPASTE system can integrate large foreign genes (donor DNA) into target sites of the intracellular genome with significantly higher efficiency than existing technologies, without the need for DNA double-strand break repair processes, thereby completing the present disclosure.

### SUMMARY

An aspect of the present disclosure is to provide a PE-Pa01NT fusion protein including a Prime Editor protein including a Cas nickase and reverse transcriptase (RT), and an integrase.

Another aspect of the present disclosure is to provide a polynucleotide including a nucleotide sequence encoding the PE-Pa01INT fusion protein or a vector including the polynucleotide.

Another aspect of the present disclosure is to provide a composition for genome editing including the PE-Pa01INT fusion protein, a polynucleotide including a nucleotide sequence encoding the PE-Pa01INT fusion protein or a vector including the polynucleotide.

Another aspect of the present disclosure is to provide a kit for genome editing including the composition.

Another aspect of the present disclosure is to provide a method of editing the genome of an individual, including introducing the composition into a eukaryotic cell or eukaryotic organism other than a human.

Another aspect of the present disclosure is to provide a cell with a genome edited by the method of editing the genome.

Additional aspects will be set forth in part in the description which follows and, in part, will be apparent from the description, or may be learned by practice of the presented embodiments.

An aspect of the present disclosure provides a PE-Pa01INT fusion protein wherein Pa01 Integrase (Pa01INT) protein is linked to Prime Editor (PE) protein. Specifically, an aspect of the present disclosure provides a PE-Pa01NT fusion protein including a Prime Editor protein including a Cas nickase and reverse transcriptase (RT), and an integrase.

The PE protein may include the amino acid sequence of SEQ ID NO: 1. The Pa01INT protein may include the amino acid sequence of SEQ ID NO: 2. The PE-Pa01INT fusion protein may be a fusion of the Prime Editor (PE) protein and the integrase (Pa01INT protein) connected by a 6×GGS linker (GGSGGSGGSGGSGGSGGS: SEQ ID NO: 3).

The PE-Pa01INT fusion protein may include the amino acid sequence of SEQ ID NO: 4. The PE-Pa01INT fusion protein may be a recombinant protein expressed from a vector including a polynucleotide encoding the PE-Pa01NT fusion protein, but is not limited thereto.

As used herein, the term “recombination” may refer to genetic recombination, and the literal definition of genetic recombination is the process in which a new gene fragment (DNA fragment) is transplanted onto a native gene inherited from parents, and in the process of gene expression, the transplanted gene segment (DNA fragment) that did not originally exist is expressed. In genetic engineering and industrial biotechnology, genetic recombination technology is applied to produce various types of substances by isolating and recombining the genes of specific cells, transplanting the recombined genes into host cells to transform them, and then producing the substances expressed by the recombined genes. The DNA created by artificially recombining gene fragments is called a recombinant DNA, and the protein expressed by the recombinant DNA in cells transformed by transplanting the recombinant DNA is called a recombinant protein.

Therefore, the PE-Pa01INT fusion protein may be produced through various methods known to those skilled in the art using the genetic recombination technology described above. For example, it can be produced by introducing a vector including a polynucleotide encoding the PE-Pa01INT fusion protein into transformed cells (e.g., bacteria, eukaryotic cells, etc.) and extracting and purifying the protein expressed from the vector, but is not limited thereto. Additionally, the PE-Pa01INT fusion protein may be produced by chemical or artificial protein synthesis methods known to those skilled in the art, but is not limited thereto.

The amino acid sequences of SEQ ID NO: 1, SEQ ID NO: 2, SEQ ID NO: 3, or SEQ ID NO: 4 may include, without limitation, not only the amino acid sequences composed of each sequence, but also amino acid sequences showing at least 80% homology, specifically at least 90%, more specifically at least 95%, even more specifically at least 98%, and most specifically at least 99% homology with the above sequences, as long as they exhibit substantially the same or corresponding efficacy as the above amino acid sequences. Additionally, it is evident that amino acid sequences with such homology, even if some sequences are deleted, modified, substituted, or added, are also within the scope of the present disclosure.

As used herein, the term “homology” refers to the degree of similarity of amino acid sequences or nucleotide sequences, which may be expressed as a percentage depending on the degree of similarity to a given amino acid sequence or nucleotide sequence. As used herein, a homologous sequence that has the same or similar activity as a given amino acid sequence or nucleotide sequence is indicated by “% homology.” For example, it can be confirmed by using standard software, specifically BLAST 2.0, to calculate parameters such as score, identity, and similarity, or by comparing sequences through hybridization experiments conducted under defined stringent conditions, and the appropriate hybridization conditions defined are within the technical scope and can be determined by methods well known to those skilled in the art (e.g., J. Sambrook et al., Molecular Cloning, A Laboratory Manual, 2nd Edition, Cold Spring Harbor Laboratory press, Cold Spring Harbor, New York, 1989; F.M. Ausubel et al., Current Protocols in Molecular Biology, John Wiley &amp; Sons, Inc., New York).

The PE protein may be a protein in which the Cas protein (CRISPR associated protein) and the reverse transcriptase (RT) protein are linked by a linker peptide.

As used herein, the term “CRISPR associated protein” or “Cas protein” refers to proteins that make up the CRISPR system and may recognize, cut, and edit specific nucleotide sequences to be used. Specifically, the Cas protein may act as genetic scissors that cut specific nucleotide sequences in the genome for genetic manipulation, such as inserting specific genes or stopping the activity of specific genes. The Cas protein may interact with one or more polynucleotides (typically guide RNA) to form a Cas protein-RNA complex.

The Cas protein is may be any of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, sCsx15, Csf1, Csf2, Csf3, Csf4, or homolog or variants thereof, but is not limited these CRISPR-associated proteins. In an embodiment, the Cas protein may be a Cas9 protein, but is not limited thereto.

As used herein, the term “Cas9 protein (CRISPR associated protein 9)” is one of the CRISPR type II RNA-guided DNA endonucleases, an RNA-guided endonuclease (RGEN), and it refers to a Cas protein responsible for the immune system of various prokaryotes. Information on the Cas9 gene and protein may be obtained from GenBank of the National Center for Biotechnology Information (NCBI). Additionally, the Cas9 protein may be derived from Streptococcus sp., such as Cas9 protein derived from Streptococcus pyogenes, Cas9 protein derived from Staphylococcus aureus, Cas9 protein derived from Campylobacter jejuni, or recombinant proteins thereof, but is not limited thereto.

In an embodiment, the Cas protein may be a Cas9 nickase. The Cas9 nickase is a variant of the amino acid sequence of the Cas9 protein, in which the mutations have occurred in some amino acids (or amino acid residues), and unlike Cas nucleases that cut both strands of DNA, cuts only one strand of DNA to form a DNA nick. Specifically, the Cas9 nickase may be Cas9 H840A nickase in which histidine, the amino acid (or amino acid residue) located at the 840th position from the N-terminus of the amino acid sequence of the Cas9 protein, is mutated to alanine, but is limited thereto.

The Cas9 H840A nickase may include the minimal amino acid sequence related to its function and may additionally include other sequences, and preferably consists of amino acid sequences known to those skilled in the art. Specifically, the Cas9 H840A nickase may include the amino acid sequence of SEQ ID NO: 21, but is not limited thereto.

As used herein, the term “reverse transcriptase (RT)” refers to an enzyme that can synthesize DNA using RNA as a template, an enzyme specifically found in retroviruses, and may also be referred to as RNA-dependent DNA polymerase.

The reverse transcriptase protein includes the minimal amino acid sequence related to its function and may additionally include other sequences, and preferably consists of amino acid sequences known to those skilled in the art. Specifically, the reverse transcriptase protein may include the amino acid sequence of SEQ ID NO: 22, but is not limited thereto.

In the PE protein, the linker peptide connecting the Cas protein and the reverse transcriptase protein may include the amino acid sequence of SEQ ID NO: 23, but is not limited thereto.

In an embodiment, the Prime Editor protein may further include a nuclear localization signal peptide. Specifically, both ends of the Prime Editor protein may be linked to a nuclear localization signal peptide. For example, an SV40 NLS peptide may be connected to one end of the Cas protein, the other end of the Cas protein and one end of the reverse transcriptase protein may be connected, and a Bp NLS peptide may be connected to the other end of the reverse transcriptase protein (see, FIG. 1).

As used herein, the term “nuclear localization signal (NLS) peptide” may refer to a specific amino acid sequence of a protein that acts as a signal for a protein synthesized in the cytoplasm to move into the nucleus. The NLS peptide may include the minimal amino acid sequence related to its function and may additionally include other sequences, and preferably consists of amino acid sequences known to those skilled in the art. Specifically, the SV40 NLS peptide may include the amino acid sequence of SEQ ID NO: 24, and the Bp NLS peptide may include the amino acid sequence of SEQ ID NO: 25, but is not limited thereto.

The NLS peptide may be connected to both ends of the PE protein by appropriate methods known to those skilled in the art. For example, click reactions (e.g., azide-alkyne chemical bonding methods including azide-DBCO click reactions), crosslinkers (e.g., polyethylene glycol (PEG)), linkers (e.g., peptides), functional groups, chemical reactions, bonding reactions, or any other methods to connect the NLS peptide to both ends of the PE protein may be used, and these can be appropriately selected by those skilled in the art.

The Pa01 Integrase (Pa01INT) protein may be an integrase protein derived from Pseudomonas aeruginosa (Pa01).

The PE-Pa01INT fusion protein operates within the cell along with guide RNA (e.g., pegRNA (prime editing guide RNA)) to insert or integrate foreign genes (i.e., external genes or target genes), specifically donor DNA, into target sites of the intracellular genome, thereby implementing the Enhanced Programmable Addition via Site-specific Targeting Elements (EPASTE) system.

Another aspect of the present disclosure provides a polynucleotide including a nucleotide sequence encoding the PE-Pa01INT fusion protein or a vector including the polynucleotide.

The PE-Pa01INT fusion protein is as described above.

The nucleotide sequence encoding the PE-Pa01INT fusion protein may include the nucleotide sequence encoding the Prime Editor protein, the nucleotide sequence encoding the 6×GGS linker, and the nucleotide sequence encoding the Pa01 Integrase (Pa01INT) protein. Specifically, the nucleotide sequence encoding the PE protein may include the nucleotide sequence of SEQ ID NO: 5. The nucleotide sequence encoding the 6×GGS linker may include the nucleotide sequence of SEQ ID NO: 6. The nucleotide sequence encoding the Pa01INT protein may include the nucleotide sequence of SEQ ID NO: 7. More specifically, the nucleotide sequence encoding the PE-Pa01INT fusion protein may include the nucleotide sequence of SEQ ID NO: 8.

The nucleotide sequences or amino acid sequences of SEQ ID NO: 5, SEQ ID NO: 6, SEQ ID NO: 7, SEQ ID NO: 8, SEQ ID NO: 21, SEQ ID NO: 22, SEQ ID NO: 23, SEQ ID NO: 24, or SEQ ID NO: 25 include, without limitation, not only the sequences composed of each sequence, but also sequences showing at least 80% homology, specifically at least 90%, more specifically at least 95%, even more specifically at least 98%, and most specifically at least 99% homology with the above sequences, as long as they exhibit substantially the same or corresponding efficacy as the above sequences. Additionally, it is evident that sequences with such homology, even if some sequences are deleted, modified, substituted, or added, are also within the scope of the present disclosure.

As used herein, the term “polynucleotide” refers to a polymer of nucleotides in which nucleotide monomers are connected in a long chain by covalent bonds. The polynucleotide may be single-stranded (coding sequence or antisense sequence) or double-stranded, and may be a DNA molecule (e.g., genome, cDNA, or synthetic DNA) or an RNA molecule (e.g., mRNA molecule) of a certain length or longer. Additionally, additional coding or non-coding sequences may be present within the polynucleotide. For example, the polynucleotide may include a poly (A) tail sequence at the end (e.g., 3′ end). Moreover, the polynucleotide may be connected to other molecules and/or support materials.

The polynucleotide may further include regulatory nucleic acid sequences operably linked to the target nucleic acid sequence for replication and/or expression (e.g., origin of replication sequences, promoter sequences, enhancer sequences, silencer sequences, etc.).

As used herein, the term “promoter” refers to a DNA molecule to which RNA polymerase binds to initiate DNA transcription. The promoter may be suitable for in vivo transcription, in vitro transcription, or transformation. For example, the promoter may be a T7 promoter, SP6 promoter, CaMV 35S promoter, actin promoter, ubiquitin promoter, pEMU promoter, MAS promoter, lacZ promoter, histone promoter, or CMV promoter, but is not limited thereto.

The polynucleotide may further include a nucleotide sequence of a ribosome binding site (RBS) operably linked to the target nucleic acid sequence for expression, where a ribosome binds to the RNA to initiate the biosynthesis of a protein from the RNA.

The polynucleotide may be modified to replicate and/or express proteins, peptides, RNA, etc., and may include a part of the genome, extra-genomic sequences, regulatory nucleic acid sequences operably linked to the target nucleic acid sequence, sequences encoded in plasmids, or smaller modified gene fragments. These fragments may be naturally isolated or synthetically modified by human hands.

The polynucleotide is not specifically limited to particular sequences as long as it encodes the target protein, peptide, or RNA for expression, and any combination of nucleotide sequences (nucleic acid sequences) is available.

Regardless of the length of the coding sequence itself, the polynucleotide can be combined with other DNA sequences such as promoters, polyadenylation signals, additional restriction enzyme sites, multiple cloning sites, and other coding segments, so its overall length can vary significantly. Therefore, polynucleotide fragments of almost any length may be applied, and the overall length may be limited to facilitate production and use for the intended purpose. Moreover, due to the degeneracy of the genetic code, it is evident to those skilled in the art that many nucleotide sequences exist that encode the proteins or peptides described herein. Some of these polynucleotides may have minimal homology to the nucleotide sequences of any natural gene. Nevertheless, due to differences in codon usage, other polynucleotides (e.g., polynucleotides optimized for human and/or primate codon preferences) may be specifically considered by the present disclosure.

The polynucleotide may be prepared, manipulated, and/or expressed using any of the well-established techniques known and available to those skilled in the art. For example, polynucleotide sequences encoding the proteins, peptides, RNA, or their functional equivalents of the present disclosure may be used within recombinant DNA molecules (e.g., vectors) aimed at expressing the proteins, peptides, RNA, or their functional equivalents within appropriate host cells. Due to the inherent degeneracy of the genetic code, other polynucleotide sequences that encode substantially the same or functionally equivalent amino acid sequences may be produced and used for cloning and/or expressing the proteins, peptides, RNA, or their functional equivalents of the present disclosure.

As used herein, the term “vector” may refer to a DNA construct that includes a nucleotide sequence including nucleotide sequences coding for a target gene operably linked to suitable regulatory sequences to replicate and/or express the target gene in a suitable host. The regulatory sequences may include promoters that initiate transcription, any operator sequences for regulating such transcription, sequences coding for appropriate mRNA ribosome binding sites, sequences for regulating the termination of transcription and translation, etc. After being transformed into a suitable host, the vector may replicate or function independently of the host genome or be integrated into the genome itself. The promoter is as described above.

The vector may be any that can be introduced into a host cell and replicate within the cell. For example, the vector may be a plasmid, cosmid, virus, replicon, or bacteriophage in its natural or recombinant state. Preferably, the vector may be a viral vector (e.g., lentivirus vector, adenovirus vector, etc.) that facilitates the entry of the target gene into the nucleus of the host cell.

The target gene included in the vector may be a nucleotide sequence coding for the target protein, peptide, or RNA, and may be the polynucleotide.

The vector may include one or more selection markers to select host cells including the vector. Selection markers may be used to confer selectable phenotypes such as drug resistance, nutritional requirements, resistance to cell toxins, or expression of surface proteins, to select cells transformed with the vector. For example, the marker may be one or more selection markers selected from the group consisting of neomycin, puromycin, hygromycin, and zeocin.

The vector may include genes involved in replication and/or copy number control, such as origins of replication and promoters, and may include restriction enzyme sites (e.g., multiple cloning sites), but is not limited thereto.

The vector may be prepared with modifications suitable for the content of the disclosure based on genetic recombination techniques known to those skilled in the art. To express the desired protein, peptide, or RNA, a nucleotide sequence encoding the desired protein, peptide, or RNA may be inserted into an appropriate expression vector (i.e., a vector including elements necessary for transcription or transcription and translation of the inserted coding sequence). In other words, an expression vector including the sequence encoding the target protein, peptide, or RNA, and appropriate transcription control elements and/or translation control elements may be constructed using methods well known to those skilled in the art. These methods may include in vitro recombinant DNA techniques, synthetic techniques, in vivo genetic recombination techniques, etc., and are described in the following references: Sambrook et al., Molecular Cloning, A Laboratory Manual (1989) and Ausubel et al., Current Protocols in Molecular Biology (1989).

The polynucleotide or vector may further include additional sequences to enhance stability, efficiency of introduction into cells (or nuclei), transcription and/or translation efficiency, RNA processing efficiency, nuclear export efficiency of transcribed RNA, gene editing efficiency, etc. Additionally, the polynucleotide or vector may further include sequences for marker substances (e.g., fluorescent substances) to confirm the expression of the target substance, i.e., the desired protein, peptide, or RNA. Furthermore, the polynucleotide or vector may have an unrestricted form, such as circular or linear.

The vector may include not only the polynucleotide including nucleotide sequences encoding the PE-Pa01INT fusion protein but also a polynucleotide including a DNA nucleotide sequence encoding guide RNA (e.g., pegRNA). Therefore, the vector may express the PE-Pa01INT fusion protein and the guide RNA. The guide RNA is described below.

Terms or elements mentioned in the context of the polynucleotide or vector that are the same as those mentioned in the description of the fusion protein are understood to be as described earlier in the context of the fusion protein.

Another aspect of the present disclosure provides a composition for genome editing, including the PE-Pa01INT fusion protein, a polynucleotide including a nucleotide sequence encoding the same, or a vector including the polynucleotide.

The PE-Pa01INT fusion protein, a polynucleotide including a nucleotide sequence encoding the same, or a vector including the polynucleotide are as described above.

The intracellular genome editing may include inserting or integrating foreign genes (i.e., donor DNA) at specific sites of the intracellular genome or modifying or deleting specific genes.

As used herein, the term “genome editing” may include the meanings of terms such as gene manipulation, gene regulation, gene correction, gene rearrangement, gene recombination, gene insertion, gene integration, and may be used interchangeably with these terms in the present disclosure.

The composition may further include guide RNA, a polynucleotide including a nucleotide sequence (i.e., DNA sequence) encoding the guide RNA, or a vector including the polynucleotide.

As used herein, the term “guide RNA” refers to target DNA-specific RNA (e.g., RNA that can hybridize with the target region of DNA) that can form a complex with Cas protein and recognizes the target DNA to induce the Cas protein to cut the target DNA region.

The guide RNA may include two guide RNAs: CRISPR RNA (crRNA) with a nucleotide sequence capable of hybridizing with the target site in the genome and trans-activating crRNA (tracrRNA) with a nucleotide sequence capable of hybridizing with the Cas protein, and it may be in the form of dual guide RNA (crRNA-tracrRNA complex) where crRNA and tracrRNA are partially combined or in the form of single guide RNA (sgRNA) where the ends of crRNA and tracrRNA are connected. Any guide RNA may be used if it includes essential parts of crRNA and tracrRNA and nucleotide sequences complementary to the target gene.

In an embodiment, the guide RNA may be pegRNA (prime editing guide RNA). The pegRNA may be composed of two types of pegRNA. Each of the two types of pegRNA hybridizes with each of the two independent Cas nickases, and target different regions in a specific gene site within the cell to be edited, inducing single-strand DNA cuts at each target site by the hybridized Cas nickases. Additionally, each of the two types of pegRNA may function as a template for new DNA synthesis by reverse transcriptase, inducing the insertion of new DNA (e.g., attB sequence) into each cut single-strand DNA. Therefore, the pegRNA may have the general configuration, form, and structure of pegRNA known in the art, may be prepared by general methods known in the art, and may be designed by changing specific sequences according to the target gene and the desired insertion gene. Specifically, the pegRNA may include a Cas protein (e.g., Cas nickase) binding site sequence, a target gene binding site sequence, a primer binding site (PBS) sequence that hybridizes with the 3′ end of the cut single-strand DNA, and a reverse transcriptase template (RTT) sequence including the genetic information to be newly synthesized into the cut single-strand DNA. The pegRNA may include the attB sequence or all or part of the complementary sequence as the genetic information to be newly synthesized in the RTT sequence.

The guide RNA (e.g., pegRNA) may further include additional sequences to enhance stability, efficiency of introduction into cells (or nuclei), target gene recognition or hybridization efficiency, hybridization efficiency with Cas proteins, gene editing efficiency, etc.

In an embodiment, the pegRNA may be two types of pegRNA targeting intron 1 of the mouse albumin gene, and in this case, the two types of pegRNA may include pegRNA including the nucleotide sequence of SEQ ID NO: 9 and pegRNA including the nucleotide sequence of SEQ ID NO: 10.

The guide RNA (e.g., one or more types of pegRNA) may include attB sequences including the nucleotide sequence of SEQ ID NO: 16 and/or SEQ ID NO: 17. In an embodiment, each of the two types of pegRNA targeting intron 1 of the mouse albumin gene may include an attB sequence including the nucleotide sequence of SEQ ID NO: 16 or an attB sequence including the nucleotide sequence of SEQ ID NO: 17. Specifically, the pegRNA including the nucleotide sequence of SEQ ID NO: 9 may include an attB sequence including the nucleotide sequence of SEQ ID NO: 16, and the pegRNA including the nucleotide sequence of SEQ ID NO: 10 may include an attB sequence including the nucleotide sequence of SEQ ID NO: 17.

In the polynucleotide (e.g., DNA) including the nucleotide sequence encoding the guide RNA or the vector including the polynucleotide, the general description of the polynucleotide or vector is as described above. However, in the case of a polynucleotide including a nucleotide sequence encoding the guide RNA or a vector including the polynucleotide, the purpose may be solely the transcription of the guide RNA from the polynucleotide including the nucleotide sequence encoding the guide RNA (e.g., DNA), without the translation process for synthesizing proteins or peptides from the transcribed RNA. Therefore, in the case of a polynucleotide including a nucleotide sequence encoding the guide RNA or a vector including the polynucleotide, it may not include a ribosome binding site sequence to prevent the translation process from occurring from the transcribed RNA. Thus, the polynucleotide including the nucleotide sequence encoding the guide RNA or the vector including the polynucleotide may be a polynucleotide or vector for RNA expression (specifically, for transcription).

The polynucleotide or vector may include nucleotide sequences encoding two or more guide RNAs in a single polynucleotide or vector, resulting in the expression of two or more guide RNAs from a single polynucleotide or vector.

Additionally, the polynucleotide or vector may be composed of two or more types, each including a nucleotide sequence encoding a different guide RNA, resulting in the expression of two or more guide RNAs from two or more polynucleotides or vectors.

In an embodiment, the polynucleotide including the nucleotide sequence encoding the guide RNA may include a nucleotide sequence encoding a pegRNA targeting intron 1 of the mouse albumin gene. The nucleotide sequence encoding the pegRNA targeting intron 1 of the mouse albumin gene (i.e., DNA sequence) may include the nucleotide sequence of SEQ ID NO: 11 and/or SEQ ID NO: 12.

The nucleotide sequence encoding the guide RNA (e.g., one or more types of pegRNA) (i.e., DNA sequence) may include attB sequences including the nucleotide sequence of SEQ ID NO: 18 and/or SEQ ID NO: 19.

The composition may further include a polynucleotide including donor DNA (i.e., a foreign gene or target gene to be inserted or integrated into the genome within the cell) or a vector including the polynucleotide.

The polynucleotide including the donor DNA or the vector including the polynucleotide may include not only the nucleotide sequence of the donor DNA but also an attP sequence. The attP sequence may include the nucleotide sequence of SEQ ID NO: 13. The attP sequence may be derived from Pseudomonas aeruginosa (Pa01).

Additionally, the polynucleotide including the donor DNA or the vector including the polynucleotide may further include all or part of the nucleotide sequence of the target site of the intracellular genome where the donor DNA is to be inserted or integrated (e.g., all or part of the intron nucleotide sequence of the target site of the intracellular genome), poly (A) tail sequences, and plasmid backbone sequences. The entire or partial nucleotide sequence of the target site of the intracellular genome included in the polynucleotide including the donor DNA or the vector including the polynucleotide may include the nucleotide sequence of the splicing acceptor site of the target intron in the intracellular genome, and in this case, the nucleotide sequence of the splicing acceptor site may be naturally spliced after being inserted into the target gene in the intracellular genome, thereby inducing the expression of the donor DNA inserted together into the target gene.

In the polynucleotide including the donor DNA or the vector including the polynucleotide, the general description of the polynucleotide or vector is as described above. However, in the case of a polynucleotide including the donor DNA or a vector including the polynucleotide, the purpose may be to insert or integrate the donor DNA itself into a specific site in the genome within the cell, and it may not be intended for transcription and translation processes to occur from the polynucleotide or vector. Therefore, in the case of a polynucleotide including the donor DNA or a vector including the polynucleotide, it may not include a promoter sequence initiating transcription (promoterless), and it may not include sequences for transcription and translation, such as ribosome binding site sequences, in addition to promoter sequences. Thus, the polynucleotide including the donor DNA or the vector including the polynucleotide may be a polynucleotide or vector for the delivery or replication of donor DNA.

The composition may further include components for transfecting the PE-Pa01INT fusion protein, a polynucleotide including the nucleotide sequence encoding it, or a vector including the polynucleotide; and/or the guide RNA, a polynucleotide including the nucleotide sequence encoding it, or a vector including the polynucleotide; and/or the polynucleotide including the donor DNA or a vector including the polynucleotide into the cell. For example, the components for transfection may include calcium phosphate, dendrimers, cationic polymers (e.g., DEAE-dextran, polyethyleneimine, etc.), or liposomes, but are not limited thereto.

In an embodiment, when the PE-Pa01INT fusion protein included in the composition, a polynucleotide including the nucleotide sequence encoding it, or a vector including the polynucleotide; and the pegRNA, a polynucleotide including the nucleotide sequence encoding it, or a vector including the polynucleotide are transfected into the cell, a new DNA sequence, attB sequence, may be inserted at the target site in the intracellular genome targeted by the pegRNA. The attB sequence inserted into the intracellular genome may include the nucleotide sequence of SEQ ID NO: 14. Additionally, in an embodiment, when the polynucleotide including the donor DNA or the vector including the polynucleotide included in the composition is transfected into a cell with the attB sequence inserted at the target site in the genome, the attP sequence included in the polynucleotide including the donor DNA or the vector including the polynucleotide, the attB sequence inserted into the target site of the intracellular genome, and the interaction of the Pa01 Integrase (Pa01INT) protein of the PE-Pa01INT fusion protein may integrate the donor DNA into the target site of the intracellular genome where the attB sequence is inserted. Through this process, the EPASTE system of the present invention may be implemented.

In an embodiment, the EPASTE system may integrate donor DNA into the target site of the intracellular genome with significantly higher efficiency compared to conventional PASTE v3 and PASTE v4 systems by using the PE-Pa01INT fusion protein in which the Pa01 Integrase is linked to the PE protein, and even if the donor DNA is large, it may be integrated into the target site of the intracellular genome with high efficiency.

The nucleotide sequences of SEQ ID NO: 9, SEQ ID NO: 10, SEQ ID NO: 11, SEQ ID NO: 12, SEQ ID NO: 13, SEQ ID NO: 14, SEQ ID NO: 16, SEQ ID NO: 17, SEQ ID NO: 18, or SEQ ID NO: 19 include, without limitation, not only the nucleotide sequences composed of each sequence, but also nucleotide sequences showing at least 80% homology, specifically at least 90%, more specifically at least 95%, even more specifically at least 98%, and most specifically at least 99% homology with the above sequences, as long as they exhibit substantially the same or corresponding efficacy as the above nucleotide sequences. Additionally, it is evident that nucleotide sequences with such homology, even if some sequences are deleted, modified, substituted, or added, are also within the scope of the present disclosure.

In (1) the PE-Pa01INT fusion protein, a polynucleotide including the nucleotide sequence encoding it, or a vector including the polynucleotide; (2) the guide RNA, a polynucleotide including the nucleotide sequence encoding it, or a vector including the polynucleotide; and (3) the polynucleotide including the donor DNA or a vector including the polynucleotide, which are included in the composition, the molar concentrations of (1) and (3) may be higher than the molar concentration of (2). For example, based on the total molar amounts of (1), (2), and (3) in the composition, the molar ratios of (1) and (3) may be higher than the molar ratio of (2).

In an embodiment, when the molar concentrations of (1) and (3) in the composition are higher than the molar concentration of (2) (or based on the total molar amounts of (1), (2), and (3) in the composition, when the molar ratios of (1) and (3) are higher than the molar ratio of (2)), the efficiency of integrating donor DNA into the target site of the intracellular genome using the EPASTE system may be significantly increased.

Terms or elements mentioned in the context of the composition that are the same as those mentioned in the description of the fusion protein or the polynucleotide or vector are understood to be as described earlier in the context of the fusion protein or the polynucleotide or vector.

Another aspect of the present disclosure provides a intracellular genome editing kit including the PE-Pa01INT fusion protein, a polynucleotide including the nucleotide sequence encoding it, or a vector including the polynucleotide. Specifically, an aspect of the present disclosure provides a genome editing kit including the composition.

The kit may further include the guide RNA, a polynucleotide including the nucleotide sequence encoding it (i.e., DNA sequence), or a vector including the polynucleotide; and/or a polynucleotide including the donor DNA or a vector including the polynucleotide.

The kit may further include (1) the PE-Pa01INT fusion protein, a polynucleotide including the nucleotide sequence encoding it, or a vector including the polynucleotide; and/or (2) the guide RNA, a polynucleotide including the nucleotide sequence encoding it, or a vector including the polynucleotide; and/or (3) a polynucleotide including the donor DNA or a vector including the polynucleotide, as well as components for transfecting (e.g., transducing) them into the cell.

In the kit, as described in items (1), (2), and (3) included therein, the molar concentrations of (1) and (3) may each be higher than the molar concentration of (2). For example, based on the total molar amounts of (1), (2), and (3) included in the kit, the molar ratios of (1) and (3) may be higher than the molar ratio of (2). In this case, the efficiency of integrating donor DNA into the target site in the genome within the cell using the EPASTE system may be significantly increased.

Terms or elements mentioned in the kit that are the same as those mentioned in the description of the fusion protein, the polynucleotide or vector, or the composition, are understood to be as described earlier in the context of the fusion protein, the polynucleotide or vector, or the composition.

Another aspect of the present disclosure provides a method for editing the genome within cells using the PE-Pa01INT fusion protein, a polynucleotide comprising a nucleotide sequence encoding the fusion protein, or a vector comprising the polynucleotide. Specifically, an aspect of the present disclosure provides a method of editing the genome of an individual, including introducing the composition into a eukaryotic cell or a eukaryotic organism other than humans.

The method may include administering or treating the individual or isolated cells with the PE-Pa01INT fusion protein, a polynucleotide including the nucleotide sequence encoding it, or a vector including the polynucleotide.

The method may further include administering or treating the individual or isolated cells with the guide RNA, a polynucleotide including the nucleotide sequence encoding it (i.e., DNA sequence), or a vector including the polynucleotide; and/or a polynucleotide including the donor DNA or a vector including the polynucleotide.

The method may further include transfecting (e.g., transducing) (1) the PE-Pa01INT fusion protein, a polynucleotide including the nucleotide sequence encoding it, or a vector including the polynucleotide; and/or (2) the guide RNA, a polynucleotide including the nucleotide sequence encoding it, or a vector including the polynucleotide; and/or (3) a polynucleotide including the donor DNA or a vector including the polynucleotide, into the cells of the individual or the isolated cell.

In the method, each of the molar concentrations of (1) and (3) administered or treated to the individual or isolated cells may be higher than the molar concentration of (2) administered or treated to the individual or isolated cells. For example, in the method, based on the total molar amounts of (1), (2), and (3) administered or treated to the individual or isolated cells, the molar ratios of (1) and (3) administered or treated to the individual or isolated cells may be higher than the molar ratio of (2) administered or treated to the individual or isolated cells. In this case, the efficiency of integrating donor DNA into the target site in the intracellular genome using the EPASTE system may be significantly increased.

The individual may include humans, non-human animals, or plants, and the non-human animals may include vertebrates such as mammals, birds, reptiles, amphibians, and fish; arthropods such as crustaceans, chelicerates, and myriapods; echinoderms; mollusks; annelids; platyhelminths; and cnidarians.

The isolated cells may be eukaryotic cells or prokaryotic cells. Specifically, the isolated cells may be cells isolated from the individual, or bacterial, fungal, or algal cells, and more specifically, they may be eukaryotic cells.

Terms or elements mentioned in the method that are the same as those mentioned in the description of the fusion protein, the polynucleotide or vector, the composition, or the kit, are understood to be as described earlier in the context of the fusion protein, the polynucleotide or vector, the composition, or the kit.

Another aspect of the present disclosure provides cells into which the PE-Pa01INT fusion protein, a polynucleotide including the nucleotide sequence encoding it, or a vector including the polynucleotide has been introduced, or cells whose genome has been edited by the method of editing the genome.

The cells may further include the guide RNA, a polynucleotide including the nucleotide sequence encoding it (i.e., DNA sequence), or a vector including the polynucleotide; and/or a polynucleotide including the donor DNA or a vector including the polynucleotide. As a result, the cells may be transgenic with the donor DNA integrated into the target site of the intracellular genome targeted by the guide RNA.

The transgenic cells or cells may be eukaryotic or prokaryotic cells. Specifically, the transgenic cells or cells may be cells isolated from animals or plants, or bacterial, fungal, or algal cells. More specifically, the transgenic cells or cells may include bacterial, fungal, virus, archaeal cells, unicellular eukaryotic organisms, plant cells (or plant-derived eukaryotic cells), algal cells (such as Botryococcus braunii, Chlamydomonas reinhardtii, Nannochloropsis gaditana, Chlorella pyrenoidosa, Sargassum patens, and C. agardh), animal cells (or animal-derived eukaryotic cells), invertebrate animal cells (e.g., insect, cnidarian, echinoderm, nematode), vertebrate cells (e.g., fish, amphibian, reptile, bird, mammal), mammalian cells (rodent cells, human cells (e.g., immune cells), non-human primate cells), but are not limited thereto.

Terms or elements mentioned in the context of the cells that are the same as those mentioned in the description of the fusion protein, the polynucleotide or vector, the composition, the kit, or the method, are understood to be as described earlier in the context of the fusion protein, the polynucleotide or vector, the composition, the kit, or the method.

According to an aspect of the present disclosure, when using the Enhanced Programmable Addition via Site-specific Targeting Elements (EPASTE) system with the Prime Editor (PE) protein and the fusion protein of Pa01 Integrase, a large donor DNA may be integrated into the target site of the intracellular genome with significantly higher efficiency compared to existing technologies, without the DNA repair process following DNA double-strand breaks.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic diagram showing the configuration of plasmids for expressing a fusion protein of Prime Editor (PE) protein and Pa01 Integrase (Pa01INT) (PE-Pa01INT fusion protein), of an EPASTE system according to an embodiment, a fusion protein of PE protein and Bxbl Integrase, of the PASTE v3 system, and a fusion protein of PE protein and Bcel Integrase, of the PASTE v4 system;

FIG. 2 is a schematic diagram showing the configuration of a promoterless plasmid for donor DNA delivery of the EPASTE system according to an embodiment;

FIG. 3 is a diagram showing the results of flow cytometry analyzing the proportion of cells expressing mCherry protein among untreated mouse cells, which are the control group to which the EPASTE system according to an embodiment is not applied (P1: mouse cell; P2: singlets; P3: singlets; P4: mCherry-positive cell (cell expressing mCherry));

FIG. 4 is a diagram showing the results of flow cytometry analyzing the proportion of cells expressing mCherry protein encoded by donor DNA among mouse cells to which the PASTE v3 system is applied to integrate foreign genes (donor DNA) into the target site of the intracellular genome, according to an embodiment (P1: mouse cell; P2: singlets; P3: singlets; P4: mCherry-positive cell (cell expressing mCherry));

FIG. 5 is a diagram showing the results flow cytometry analyzing the proportion of cells expressing mCherry protein encoded by donor DNA among mouse cells to which the PASTE v4 system is applied to integrate foreign genes (donor DNA) into the target site of the intracellular genome, according to an embodiment (P1: mouse cell; P2: singlets; P3: singlets; P4: mCherry-positive cell (cell expressing mCherry)); and

FIG. 6 is a diagram showing the results of flow cytometry analyzing the proportion of cells expressing mCherry protein encoded by donor DNA among mouse cells to which the EPASTE system according to an embodiment is applied to integrate foreign genes (donor DNA) into the target site of the intracellular genome (P1: mouse cell; P2: singlets; P3: singlets; P4: mCherry-positive cell (cell expressing mCherry)). This shows the efficiency of integration of the foreign gene (donor DNA) into intron 1 of a mouse albumin gene, of the EPASTE system according to an embodiment.

### DETAILED DESCRIPTION

Reference will now be made in detail to embodiments, examples of which are illustrated in the accompanying drawings, wherein like reference numerals refer to like elements throughout. In this regard, the embodiments may have different forms and should not be construed as being limited to the descriptions set forth herein. Accordingly, the embodiments are merely described below, by referring to the figures, to explain aspects of the description. As used herein, the term “and/or” includes any and all combinations of one or more of the associated listed items. Expressions such as “at least one of,” when preceding a list of elements, modify the entire list of elements and do not modify the individual elements of the list.

### EXAMPLE 1. DEVELOPMENT OF THE ENHANCED PROGRAMMABLE ADDITION VIA SITE-SPECIFIC TARGETING ELEMENTS (EPASTE) SYSTEM

An EPASTE (Enhanced Programmable Addition via Site-specific Targeting Elements) system was developed for inserting foreign genes into specific regions of the intracellular genome (e.g., DNA). The EPASTE system mainly consists of the PE-Pa01INT fusion protein, prime editing guide RNA (pegRNA), and the donor DNA to be inserted.

Specifically, three types of plasmids were prepared to express or deliver each of the PE-Pa01INT fusion protein, pegRNA (prime editing guide RNA), and the donor DNA to be inserted. First, the PE-Pa01INT fusion protein is a protein where the Prime Editor protein, having the amino acid sequence of SEQ ID NO: 1, is linked to Pa01 Integrase (Pa01INT) with the amino acid sequence of SEQ ID NO: 2 by a 6×GGS linker (GGSGGSGGSGGSGGSGGS: SEQ ID NO: 3), and this can be obtained via a plasmid expressing this fusion protein.

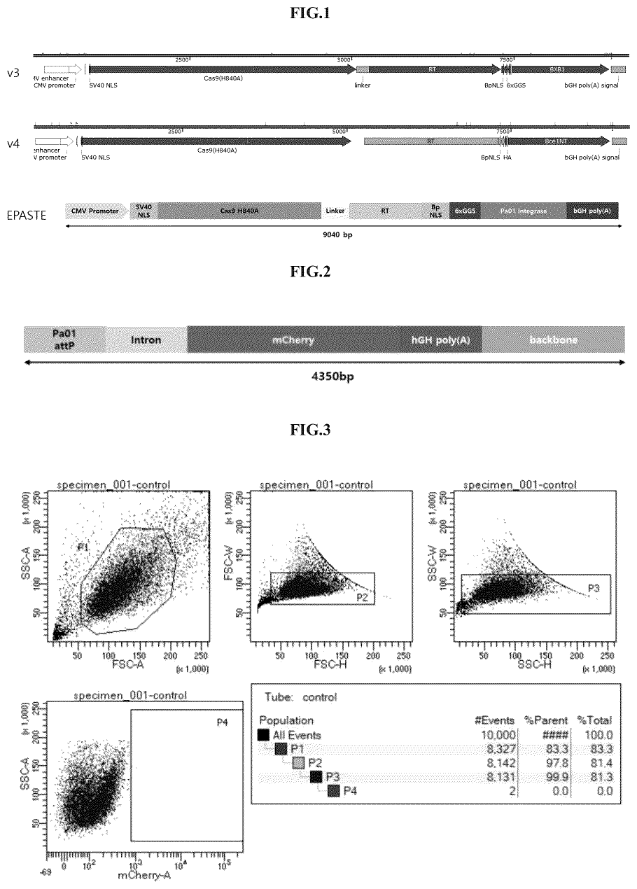

Specifically, the PE protein is a protein where the Cas9 (CRISPR associated protein 9) H840A nickase protein (SEQ ID NO: 21) and the reverse transcriptase (RT) protein (SEQ ID NO: 22) are linked by a linker peptide (SEQ ID NO: 23), and the two termini of the PE protein are connected to nuclear localization signal (NLS) peptides (for example, the SV40 NLS peptide (SEQ ID NO: 24) is connected to one terminus of the Cas9 H840A nickase protein, and the Bp NLS peptide (SEQ ID NO: 25) is connected to one terminus of the RT protein). A plasmid for expressing the PE-Pa01INT fusion protein was prepared, including the nucleotide sequence encoding this PE protein (SEQ ID NO: 5), the nucleotide sequence encoding the 6×GGS linker (SEQ ID NO: 6), and the nucleotide sequence encoding Pa01 Integrase (Pa01INT) (SEQ ID NO: 7) (FIG. 1).

Next, a plasmid for expressing pegRNA targeting intron 1 of the mouse albumin gene was prepared. The pegRNA consists of two types of pegRNA, namely, a pegRNA including the nucleotide sequence of SEQ ID NO: 9 and a pegRNA including the nucleotide sequence of SEQ ID NO: 10. Each of the pegRNAs targets a specific genomic region within the cell to be edited, inducing single-strand DNA cuts at the target site by Cas9 H840A nickase and acting as a template for new DNA synthesis by reverse transcriptase, thereby guiding the insertion of new DNA (e.g., attB sequence) into the cut single-strand DNA. Therefore, the pegRNA is suitable for the aforementioned function, possessing the general composition, form, and structure known in the art for pegRNAs, and can be designed by modifying specific sequences according to the target gene or to increase gene editing efficiency. Specifically, each of the pegRNAs includes a Cas9 H840A nickase binding site sequence, an intron 1 of the mouse albumin gene binding site sequence (target gene region), a primer binding site (PBS) sequence that hybridizes with the 3′ end of the cut single-strand DNA, and a reverse transcriptase template (RTT) sequence including the genetic information to be newly synthesized into the cut single-strand DNA, where the RTT sequence is designed to include all or part of the attB sequence or its complementary sequence as the newly synthesized genetic information. Two plasmids were prepared to express these two types of pegRNAs targeting intron 1 of the mouse albumin gene, namely, a plasmid including the DNA nucleotide sequence encoding one type of pegRNA (SEQ ID NO: 11) and a plasmid including the DNA nucleotide sequence encoding the other type of pegRNA (SEQ ID NO: 12). The DNA nucleotide sequence encoding one type of pegRNA (SEQ ID NO: 11) includes an attB sequence including the nucleotide sequence of SEQ ID NO: 18, and the DNA nucleotide sequence encoding the other type of pegRNA (SEQ ID NO: 12) includes an attB sequence including the nucleotide sequence of SEQ ID NO: 19.

Additionally, a promoterless plasmid for delivering the donor DNA to be inserted was prepared. Specifically, a promoterless plasmid for delivering donor DNA was prepared, including the Pa01-attP sequence with the nucleotide sequence of SEQ ID NO: 13, part of intron 1 of the mouse albumin gene sequence (including splicing acceptor sequence) (SEQ ID NO: 20), the mCherry protein coding sequence (target DNA for insertion) with the nucleotide sequence of SEQ ID NO: 15, hGh poly (A) sequence, and plasmid backbone sequence (FIG. 2). The part of intron 1 of the mouse albumin gene sequence (including splicing acceptor sequence) included in the promoterless plasmid for delivering donor DNA is naturally spliced after being inserted into the target gene of the intracellular genome, thereby inducing the expression of the target DNA (i.e., mCherry protein coding sequence) inserted together into the target gene.

When the three prepared plasmids (the plasmid for expressing the PE-Pa01INT fusion protein, the plasmid for expressing pegRNA, and the promoterless-mCherry plasmid for delivering donor DNA) are transfected into cells, the PE-Pa01INT fusion protein and the two types of pegRNA are expressed from these plasmids within the cells through the intracellular gene expression system. Each of the expressed PE-Pa01INT fusion proteins binds to one type of pegRNA, forming two types of complexes in total, and the pegRNA of each complex binds to the target gene region of the intracellular DNA (e.g., intron 1 of the mouse albumin gene), guiding the Cas9 H840A nickase of the PE-Pa01INT fusion protein to cut the single-stranded DNA at the target gene region. At this time, the two complexes cut separate single strands of DNA at the target gene region by each pegRNA. The 3′ end of each cut single-strand DNA binds to the PBS sequence of each pegRNA, and the reverse transcriptase of the PE-Pa01INT fusion protein uses the RTT sequence located after the PBS sequence of the pegRNA as a template to extend the 3′ end of each cut single-strand DNA, synthesizing a new DNA sequence, specifically the attB sequence. Subsequently, the new DNA sequence, that is, attB sequence, is synthesized and annealed between single DNA strands to form a 3′ flap, while the original sequence without the new DNA sequence is annealed between single DNA strands to form a 5′ flap, and the cellular repair system excises the 5′ flap, and only the 3′ flap including the new DNA sequence, specifically the attB sequence, is ligated to the genomic DNA. As a result, the target gene region of the intracellular DNA is in a state where the new DNA sequence (i.e., the attB sequence: SEQ ID NO: 14) is stably inserted. Subsequently, through the interaction of the attB sequence inserted in the target gene region of the intracellular DNA, the Pa01 Integrase (Pa01INT) of the PE-Pa01INT fusion protein, and the Pa01-attP sequence included in the promoterless plasmid for delivering donor DNA that has been introduced into the cell, and the donor DNA (i.e., target DNA for insertion) included in the promoterless plasmid is inserted into the target gene region of the intracellular DNA where the attB sequence has been inserted, thereby achieving integration. Through this process, the EPASTE system operates to insert and integrate foreign genes into specific regions of the intracellular genome, and with the EPASTE system, large donor DNA can be integrated into the target region of the intracellular genome with very high efficiency, without the DNA repair process following double-strand DNA breaks. The EPASTE system can integrate foreign genes into the target region of the intracellular genome with significantly superior efficiency compared to existing technologies, as demonstrated in the following experimental examples.

### EXPERIMENTAL EXAMPLE 1. VERIFICATION OF THE INTEGRATION EFFICIENCY OF FOREIGN GENES INTO THE TARGET SITE OF THE INTRACELLULAR GENOME USING EPASTE SYSTEM

After transfecting the EPASTE system of Example 1, specifically the three plasmids (the plasmid for expressing the PE-Pa01INT fusion protein, the plasmid for expressing pegRNA, and the promoterless plasmid for delivering donor DNA) into mouse cells, the integration efficiency of the donor DNA at the target site of the intracellular genome of the mouse cells, specifically at intron 1 of the albumin gene, was analyzed through flow cytometry.

Specifically, after transfecting the mouse cells (mouse hepatocyte cell line (Hepa1c1c7)) with each of the three plasmids at the same molar concentration (with the molar ratio of the plasmid for expressing one type of pegRNA, the plasmid for expressing the other type of pegRNA, the plasmid for expressing the PE-Pa01INT fusion protein, and the promoterless plasmid for delivering donor DNA being 1:1:1:1), approximately 96 hours later, the ratio of cells expressing the mCherry protein, a red fluorescent protein encoded by the donor DNA, was measured using a flow cytometer to analyze the integration efficiency of the donor DNA by the EPASTE system of Example 1. As controls, the integration efficiency of donor DNA was analyzed in the same manner for cells without the EPASTE system (untreated cells), cells with the conventional PASTE v3 system, and cells with the conventional PASTE v4 system. The PASTE v3 system differs from the EPASTE system of Example 1 in that it uses a fusion protein linking the PE protein with Bxbl Integrase instead of Pa01 Integrase, and the PASTE v4 system uses a fusion protein linking the PE protein with Bcel Integrase instead of Pa01 Integrase.

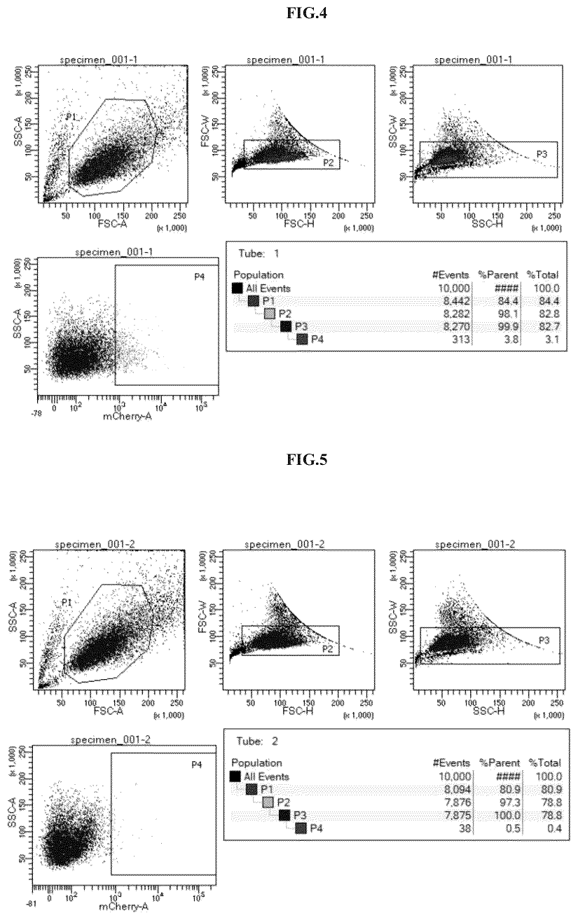

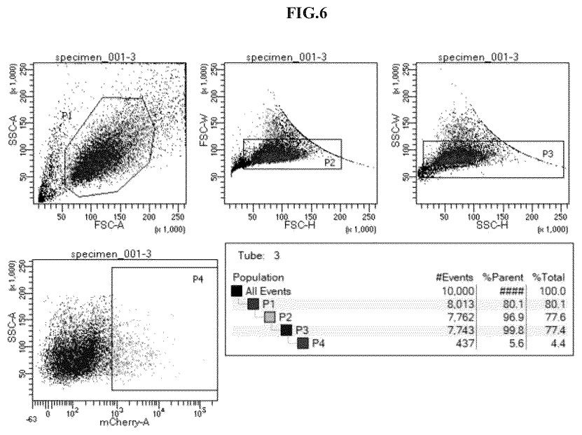

As a result, as shown in FIG. 3, the ratio of cells expressing the mCherry protein was confirmed to be 0% in the control cells, i.e., the untreated cells without the EPASTE system of Example 1 (P4 in FIG. 3). On the other hand, as shown in FIGS. 4 and 5, the ratio of cells expressing the mCherry protein encoded by the donor DNA was confirmed to be 3.8% and 0.5% in the cells with the conventional PASTE v3 system or PASTE v4 system, respectively (P4 in FIGS. 4 and P4 in FIG. 5), and as shown in FIG. 6, the ratio of cells expressing the mCherry protein encoded by the donor DNA was confirmed to be 5.6% in the cells with the EPASTE system of Example 1 (P4 in FIG. 6). These results indicate that the integration efficiency of donor DNA using the EPASTE system of Example 1 is approximately 1.4 times higher compared to the conventional PASTE v3 system and about 11 times higher compared to the conventional PASTE v4 system.

Through this experimental example, it was confirmed that the EPASTE system of Example 1, by using the PE-Pa01INT fusion protein in which Pa01 Integrase is linked to the PE protein, can specifically insert, that is, integrate foreign genes into the target site of the intracellular genome with significantly superior efficiency compared to the conventional PASTE v3 and PASTE v4 systems.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

1. A fusion protein, comprising:
a Prime Editor protein comprising a Cas nickase and a reverse transcriptase (RT); and
an integrase.

2. The fusion protein of claim 1, wherein the Prime Editor protein and the integrase are connected via a linker.

3. The fusion protein of claim 2, wherein the linker comprises the amino acid sequence of SEQ ID NO: 3.

4. The fusion protein of claim 1, wherein the Cas nickase comprises the amino acid sequence of SEQ ID NO: 21.

5. The fusion protein of claim 1, wherein the reverse transcriptase comprises the amino acid sequence of SEQ ID NO: 22.

6. The fusion protein of claim 1, wherein the integrase is Pa01 integrase (Pa01INT) derived from Pseudomonas aeruginosa (Pa01).

7. The fusion protein of claim 6, wherein the Pa01 integrase comprises the amino acid sequence of SEQ ID NO: 2.

8. The fusion protein of claim 1, wherein the Prime Editor protein further comprises a nuclear localization signal peptide.

9. A polynucleotide comprising a nucleotide sequence encoding the fusion protein of claim 1, or a vector comprising the polynucleotide.

10. A composition for genome editing, comprising:
the fusion protein of claim 1, a polynucleotide comprising a nucleotide sequence encoding the fusion protein, or a vector comprising the polynucleotide.

11. The composition of claim 10, further comprising:
a prime editing guide RNA (pegRNA), a polynucleotide comprising a nucleotide sequence encoding the pegRNA, or a vector comprising the polynucleotide.

12. The composition of claim 10, further comprising:
a polynucleotide comprising donor DNA or a vector comprising the polynucleotide.

13. The composition of claim 12, wherein the polynucleotide or the vector further comprises an attP sequence.

14. A kit for genome editing, comprising:
the composition of claim 10.

15. A method for editing the genome of an individual, comprising:
introducing the composition of claim 10 into a eukaryotic cell or a eukaryotic organism other than a human.
